(19) United States
(12) Patent Application Publication
(10) Pub. No.: US 2022/0288187 A1
(43) Pub. Date: Sep. 15, 2022

(54) BIODEGRADABLE NANOCOMPLEX VACCINES, METHODS FOR SUPPRESSION OF HEPAPITIS B VIRUS REPLICATION AND HEPAPITIS B VIRUS SURFACE ANTIGEN SECRETION

(71) Applicants: Frank Wen-Chi LEE, Bedford, MA (US); Ascendo Biotechnology, Inc., Taipei (TW)

(72) Inventors: Ping-Yen HUANG, Taipei (TW); Frank Wen-Chi LEE, Bedford, MA (US); Yu-Hung CHEN, Taipei (TW); Yan-Wei WU, Taipei (TW)

(73) Assignee: Ascendo Biotechnology, Inc., Taipei (TW)

(21) Appl. No.: 17/763,184

(22) PCT Filed: Sep. 23, 2020
(86) PCT No.: PCT/US2020/052264

§ 371 Date: Mar. 23, 2022

Related U.S. Application Data
(60) Provisional application No. 62/904,267, filed on Sep. 23, 2019.

(51) Int. Cl. A61K 39/12; A61P 31/20

(52) U.S. Cl. CPC A61K 39/12; A61P 31/20; A61K 2039/55505

(57) ABSTRACT

A hepatitis B virus (HBV) vaccine includes an HBV core antigen (HBcAg) and/or HBV surface antigen (HBsAg) formulated in nanocomplexes. The nanocomplexes contain chitosan and γ-PGA. These nanocomplexes containing HBc/sAg, chitosan, and γ-PGA can induce more balanced T helper cells (Th1 and Th2) polarization than can a conventional vaccine with an alum adjuvant. HBc/s-NC of the invention can elicit high levels of antibodies against HBsAg, a rapid elimination of HBsAg, and a slow decrease of HBeAg, indicating a phenomenon of HBsAg seroconversion. Thus, HBc/s-NC can overcome immune tolerance caused by chronic HBV infection to re-establish host immunity leading a functional cure.

### FIELD OF INVENTION

The invention relates to Hepatitis B virus (HBV) vaccines, particularly to nanocomplex vaccines.

### BACKGROUND

Hepatitis B virus (HBV) infection is an important public health issue even in 21^{st} century. People are infected by HBV through contact with contaminated body fluids, e.g., blood or semen. Vertical infection, sexual transmission, and unsafe medical behaviors are three major viral transmission routes. Serological evidence shows that 2 billion people have been infected, and more than 350 million are chronic infected by the virus. World Health Organization (WHO) also includes viral hepatitis in its major public health priorities. The outcome of acute HBV infection depends on age. Although most patients will recover from HBV infection, patients progressing to chronic infection carry HBV for almost whole life. About 95% of infants, 30% of 1-5 years old children and less than 5% of adults develop chronic infection. Chronic HBV infection will increase the risk of liver fibrosis and hepatocellular carcinoma. Therefore, HBV infection is recognized as the tenth leading cause of death worldwide.

There are 8 major HBV genotypes (A-H) in humans. Genotypes A and C are prevalent in the United States, and genotype A is prevalent in Africa. Infections in East Asia are usually genotypes B and C, and infections in Southern Europe and India are genotype D. Among these HBV genotypes, C is associated with development of liver fibrosis and an increased risk of hepatocellular carcinoma. In the HBV life cycle, the covalently closed circular DNA (cccDNA) is the structure of HBV DNA formed in hepatocyte nucleus. The cccDNA can stably persist in host nucleus to serve as a template for viral RNA transcription. In addition, HBV DNA can be integrated into host genome.

HBV proteins are translated to help the replication cycle. Some of these proteins are secreted into blood circulation and can be recognized as serological markers. HBV surface antigen (HBsAg) and envelope antigen (HBeAg) are two major biomarkers of patients with HBV current or past infections. Patients with acute HBV infections are HBeAg-positive (HBeAg⁺) but turns to negative when chronic infection develops. Using specific antibodies (HBc/e/sAb) against HBc/e/sAg (i.e., HBV core, envelope, or surface antigen), the acute or chronic phase of the infection can be clearly defined. All HBV infected people produce HBcAb, and roughly 80% of recovered people (resolved infection) produce HBsAb. Presence of HBsAg for over 6 months is defined as chronic infection. In detail, HBsAg⁺IBsAb⁺HBcAb⁺HBV DNA⁺ patients are upon HBV infection, and HBsAg⁻HBsAb⁺HBcAb⁺HBV DNA⁻ are recovered ones. Roughly 80% of infected adults develop HBsAb (termed anti-HBs seroconversion). People who have been vaccinated represent HBsAg⁻HBsAb⁺HBcAb⁻HBV DNA⁻.

Chronic HBV infection can be divided into 4 phases: HBeAg⁺ immunotolerance phase, HBeAg⁺ immune-active phase, HBeAg⁻ inactive phase, and HBeAg⁻ immuno-reactive phase. All phases are HBsAg⁺, and other serological markers used to distinguish these phases depend on the HBeAg and HBeAb, HBV DNA, level of alanine aminotransferase (ALT; a sensitive marker of liver inflammation), and the intrahepatic necroinflammation. The HBeAg⁺ immunotolerance phase is characterized as HBeAb⁻, high level of DNA, normal ALT, and mild liver inflammation. The increases of ALT and liver inflammation means patients progressing to the HBeAg-positive immune-active phase. Furthermore, HBeAg loss, HBeAb⁺ and low level of DNA represent the HBeAg⁻ inactive phase. This phase may have fibrosis from previous inflammation. Once the ALT and liver inflammation increase, patients are going into the HBeAg immuno-reactive phase. Overall, patients can progress into these phases repeatedly relating to host immunity. Medical treatments usually follow these indicators of different phases.

Obviously, chronic infection is due to parasitic presence of HBV in the host. In addition to integration of the viral genome into the host genome, immune modulation of viral proteins, especially HBsAg, plays an important role. Chronic HBV infection produces large quantities of subviral particles (SVPs) containing only surface antigens and host-derived lipid membrane. The SVPs cause a phenotype of T cell exhaustion or even depletion. In addition, HBsAg can inhibit cytokines secretion from activated macrophages and dendritic cells through the regulation of innate immunity. More HBV mutants and recently defined mechanisms of HBV-mediated immune response modulation lead to concepts for preventive and therapeutic vaccination. Several preventive HBV vaccines (HBsAg) are available currently, such as GenHevac B (Merck & Co, West Point, PH), Recombivax HB (Merck), Engerix-B (GSK), Elovac B (Human Biologicals Institute), HEPLISAV-B (Dynavax Technologies Corporation), etc. However, patients with ongoing chronic infection still need long-term medical resources and have risks of hepatoma progression. Therefore, researchers are focusing on effective treatments to cure chronic HBV infections.

HBV cures are divided into three conditions: virological cure, functional cure, and partial cure. Theoretically, the virological cure means the absence of HBV DNA in the blood circulation and liver. After treatment, functional cure is determined by HBsAg loss and an undetectable level of HBV DNA in the peripheral blood. If patients still express HBsAg at low or undetectable levels, it means a partial cure.

Current standard treatments include those using interferons (IFNs) and nucleos(t)ide analogues. There are three major IFNs: α, β and γ, which can inhibit HBV replication, and may clear cccDNA through unknown mechanisms. The usage frequencies of IFNs are restricted by its adverse effects, including cytopenia, exacerbations of neuropsychiatric symptoms (such as depression and insomnia), and induction of thyroid autoantibodies. Responses to INF treatments remain barely satisfactory, and only about one-third of patients achieve HBeAg loss and fewer achieve HBsAg loss.

On the other hand, five types of nucleos(t)ide analogues are used in the United States. They are lamivudine, adefovir, entecavir, tenofovir disoproxil, and tenofovir alafenamide. These nucleos(t)ide analogues suppress HBV infections by inhibition of RNA-dependent DNA polymerase reverse transcriptase. The treatments with nucleos(t)ide analogues can reduce the HBV DNA levels, and their adverse effects are milder than those of IFNs. However, serological responses (HBeAg and HBsAg loss with or without detection of corresponding antibodies) from treatments with nucleos(t)ide analogues were low (11%-32% and 0%-2%, respectively). Moreover, the effects of these treatments are sensitive to HBV mutations, which may occur in chronic infections. Even so, lifelong treatments with oral direct antiviral drugs are currently the most popular treatment approach recommended by most hepatologists.

Several groups tried to apply commercial prophylactic vaccines as therapies to treat chronic HBV patients. The randomized controlled trials of vaccines with/without standard of care (SC) includes GenHevac B, Yeast-derived immune complexes with HBsAg, HBVAXPro, ASO2B adjuvant with HBsAg, DNA vaccines, Sci-B-Vac, GS-4774, and ABX203. Efficacy outcomes include HBeAg seroconversion, HBV DNA reduction, and HBsAg loss. According to a meta-analysis of the efficacy of therapeutic vaccinations from these trails, there were few studies on HBsAg loss, and the findings were inconclusive. The relatively clear efficacy finding is HBV DNA reduction at the end of follow-up for therapeutic vaccines with SC vs SC only. The limited efficacy only reported in few randomized controlled trials, suboptimal therapeutic effects of the vaccine candidates, and patient selections. These therapeutic vaccines do not appear to be efficacious for the treatments of chronic HBV infections.

High levels of HBsAg play an important role in T cell exhaustion and inhibition of innate immunity. It seems that the current drugs and therapeutic vaccine candidates have poor effectiveness in seroconversions of HBsAb, which is an indicator of functional cure. Therefore, there is still a need for better vaccines that can cure HBV infections.

### SUMMARY

Embodiments of the invention relate to treatments and preventions of chronic HBV infections. Embodiments of the invention use nanocomplex vaccine technology described in U.S. Pat. No. 10,052,390 B2, EP 2754436, Chinese Patent No. CN103910892B, and Taiwan Patent No. 1511744. Briefly, HBcAg and/or HBsAg are encapsulated in nanocomplexes using a simple electro-kinetic approach by addition of a charged polymer solution into another oppositely charged polymer solution. In these embodiments, HBcAg and/or HBsAg are the encapsulated immunogens in the first charged polymer solution. The first charged polymer solution also contains poly-γ-glutamic acid (γ-PGA) with negative charges. γ-PGA are commercially available (e.g., MilliporeSigma Corporation, St. Louis, Mo., U.S.A.). Any suitable molecular weight range of γ-PGA may be used with embodiments of the invention. In preferred embodiments, γ-PGA has a weight-averaged M.W. of about 200 kDa or less. The second charged polymer solution contains chitosan (CS) with positive charges. Chitosan is available from many commercial sources. Any chitosan with a suitable molecular weight range and degree of deacetylation may be used with embodiments of the invention. In preferred embodiments, chitosan may have a weight-averaged molecular weight (MW) of about 10-100 kDa. Chitosan with such molecular weights is adapted for adequate solubility at a pH that maintains the bioactivity of protein and peptide drugs.

Embodiments of the invention may use any suitable concentrations of the antigens and nanocomplex components. Exemplary ranges of concentrations may be as follows: in the first solution: HBc/sAg: 2 to 0.5 mg/ml and γ-PGA: 5-20 mg/ml, and in the second solution: Cs: 20 to 30 mg/ml. The nanocomplexes (NCs) may have zeta potentials of from about +30 mV to about +50 mV and an adjustable size range from 100 nm to 800 nm. These positively charged HBc/s-NCs are shown to have unusual therapeutic efficacies in the prevention and treatment of HBV infections.

One aspect of the invention relates to HBV vaccines. An HBV vaccine according to one embodiment of the invention comprises HBV core antigen (HBcAg) and/or HBV surface antigen (HBc/sAg) formulated in nanocomplexes. The nanocomplexes comprise γ-polyglutamic acid (γ-PGA) and chitosan. The nanocomplexes are prepared by mixing a first charged solution containing the antigen proteins with a second charged solution. Exemplary concentrations of various components are: HBcAg and/or HBc/sAg are about 2 to 0.5 mg/ml and γ-PGA is about 5-20 mg/ml in the first charged solution, and chitosan is about 20 to 30 mg/ml in the second charged solution. The nanocomplexes have a zeta potential of about +30 mV to about +50 mV.

Another aspect of the invention relates to methods for treating or preventing HBV infections. A method in accordance with one embodiment of the invention comprises administering to a subject in need thereof a composition comprising any of the above described nanocomplex vaccine. One skilled in the art would appreciate that “treating” or “treatment” means reduction or elimination of symptoms, while “preventing” or “prevention” in the context of vaccines means induction of antibody formations or immune responses such that the disease condition does not occur or occurs to a lesser extent. A vaccine of the invention may be administered via any suitable routes, such as injections (intramuscular, subcutaneous, etc.), nasal sprays, oral, etc. An effective amount for vaccination would depend on several factors (e.g., formulation, administration routes, etc.) and one skilled in the art would be able to determine effective amounts without inventive efforts.

Other aspect of the invention would become apparent with the following detailed description and the attached drawings.

### BRIEF DESCRIPTION OF THE DRAWINGS

FIG. 1 shows HBcAg and HBsAg natural form and reduced form in SDS-page.

FIG. 2 shows the Z-average, polydisperse index (PdI), and zeta-potential of HBc/s-NCs (nanocomplexes). PdI (Polydisepersity Index) is determined by DLS (Dynamic light scattering) measurements. PDI is defined as the square of the standard deviation divided by the square of the mean.

FIG. 3 shows a schedule of vaccinations, blood samplings, and sacrifice using a C57BL/6 mice model for testing vaccines of the invention.

FIG. 4A shows the body weight change chart from day 0 to day 28, and FIG. 4B shows the weights of spleen divided by body weights at day 28. Mice were inoculated with nanocomplex (NC) only, or HBc/s-Alum (a conventional adjuvant), 20 μg/dose HBc/s-NCs, or 10 μg/dose HBc/s-NC, and the body weights of mice were monitored weekly until day 28, at which time the mice were sacrificed and the spleens were removed and weighed.

FIGS. 5A-5D show antigen-specific immunoglobulin G1 (IgG1) and G2a (IgG2a) serum levels upon inoculations with NC only, HBc/s-Alum, 20 μg/dose HBc/s-NCs, or 10 μg/dose HBc/s-NCs. FIG. 5A shows the anti-HBcAg IgG1 serum levels in the groups, and FIG. 5B shows the anti-HBcAg IgG2a serum levels in the groups. FIG. 5C shows the anti-HBsAg IgG1 serum levels in the groups, and FIG. 5D shows the anti-HBsAg IgG2a serum levels in the groups. Data are presented as mean±SD. Statistical analyses were performed with one-way ANOVA, followed by Tukey's multiple comparisons test. *p<0.05, **p<0.01, ***p<0.001.

FIG. 6 shows a plan for animal model study including a schedule of infection, vaccinations, blood samplings, and sacrifice with an AAV/HBV C57BL/6 mice model for testing efficacies of vaccines of the invention.

FIG. 7 shows the changes of alanine aminotransferase (ALT) levels in sera from AAV/HBV infected mice, which were then inoculated with saline (non-treatment, NT), 10 μg/dose HBc-NCs, or 10 μg/dose HBc/s-NC.

FIG. 8 shows the changes of bilirubin levels in sera from AAV/HBV infected mice, which were then inoculated with saline (non-treatment, NT), 10 μg/dose HBc-NC, or 10 μg/dose HBc/s-NCs.

FIG. 9 shows the results of changes in HBsAg titers in sera from the appointed groups. Sera came from AAV/HBV infected mice, which were then inoculated with saline (non-treatment, NT), 10 μg/dose HBc-NC, or 10 μg/dose HBc/s-NCs.

FIGS. 10A-10C show the changes in HBsAg titers in individual mouse from the appointed group. Sera came from AAV/HBV infected mice, which were then inoculated with saline, 10 μg/dose HBc-NCs, or 10 μg/dose HBc/s-NCs. FIG. 10A shows the results from inoculation with saline (non-treatment, NT). FIG. 10B shows result from inoculation with 10 μg/dose HBc-NCs, and FIG. 10C shows results from inoculation with 10 μg/dose HBc/s-NCs.

FIG. 11 shows the results of changes in HBeAg titers in sera from the appointed groups. AAV/HBV infected mice were inoculated with saline, 10 μg/dose HBc-NCs, or 10 μg/dose HBc/s-NCs. Then, serum samples were collected at indicated times for HBeAg titer measurements.

FIGS. 12A-12C show the changes in HBeAg titers in individual mouse from the appointed group. Sera came from AAV/HBV infected mice, which were then inoculated with saline, 10 μg/dose HBc-NCs, or 10 μg/dose HBc/s-NCs. FIG. 12A shows results from inoculation with saline (non-treatment, NT). FIG. 12B shows results from inoculation with 10 μg/dose HBc-NCs, and FIG. 12C shows results from inoculation with 10 μg/dose HBc/s-NCs.

FIG. 13 shows antibody (HBsAg specific IgG) serum levels at week 10 upon inoculations with NC only (non-treatment, NT), 10 μg/dose HBc-NCs, or 10 μg/dose HBc/s-NCs. Data are presented as mean±SD. Statistical analyses were performed with one-way ANOVA, followed by Tukey's multiple comparisons test. ***p<0.001.

### DETAILED DESCRIPTION

Chronic HBV infection is a critical medical issue worldwide. Considering the outcome of virulence factors and repeated inflammation, preventive vaccine and drug therapy are used broadly. Patients with chronic HBV infection present HBV S antigen (HBsAg), E antigen (HBeAg) and DNA as serological markers. With high levels of HBsAg, HBV can escape host immunity by inhibition of innate immunity and T cell exhaustion. Therefore, the most effective therapy needs to break the immune tolerance to revive host immunity. Inventors of the present invention found that HBV antigens formulated in nanocomplexes (NC) composed of charged polymers can induce both T helper (Th) 1 and 2 responses, thereby breaking the immune tolerance of chronic HBV infection.

Embodiments of the invention relate to HBV vaccines that can be used in the prevention and/or therapies for HBV infections. These vaccines comprise antigens in novel nanocomplexes that can elicit highly effective immune responses. Inventors of the invention found an electro-kinetic approach to preparing these nanoparticle-based vaccines. This approach is very different from the conventional vaccine technologies. This technique manipulates the electric double layers of solution systems to encapsulate proteins with (+/−)-charged polymers by compressive force to form a stable, narrow charge-distribution, and dispersive spherical nanocomplex (cf. U.S. Pat. No. 10,052,390 B2; EU: 2754436; China: CN103910892B; Taiwan: 1511744; the disclosures of all these patents are incorporated by reference in their entirety).

Inventors of the invention unexpectedly found that the antigens (Ag) encapsulated in nanocomplexes (NC) can induce balanced T helper (Th) 1 and 2 immune responses and maintain long-term antibody (Ab) productions. These antigen nanocomplexes are found to elicit stronger and more comprehensive immune responses than those induced by antigens with conventional adjuvants (e.g., alum). Due to stronger and more comprehensive immune responses, these nanocomplex vaccines containing HBV antigens were unexpectedly found to be able to break the immune tolerance in chronic HBV infections.

Vaccines of the invention may use commercially available HBV antigen proteins or recombinant proteins or fragments thereof. Based on the known sequences, production of these antigen proteins (HBcAg or HBsAg) may use any suitable techniques known in the art. For example, HBsAg produced in yeast cells is used by Merck to produce an HBV vaccine, Recombivax HB®. In accordance with embodiments of the invention, the antigen proteins may be full-length HBcAg or HBsAg, or immunogenic fragments thereof.

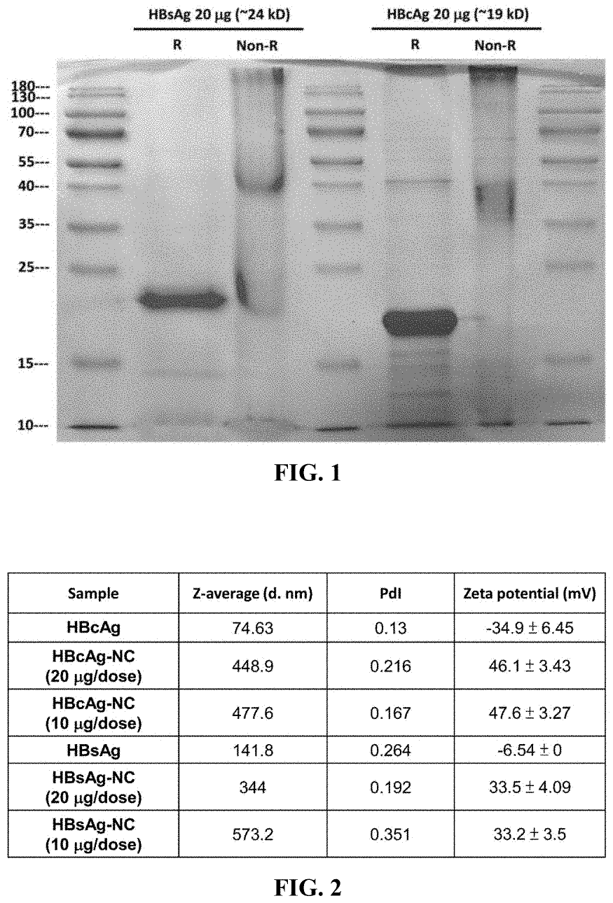

In the following examples, full-length HBcAg and HBsAg produced with recombinant technology are used. To evaluate the purities and stabilities of HBc/sAg proteins, non-reduced proteins or proteins reduced with beta-mercaptoethanol (2-ME) or dithiothreitol (DTT) were loaded on SDS-page. After electrophoresis, these proteins were separated to relative locations depending on their MWs (HBc about 19 kDa; HBs about 24 kDa). Coomassie Blue staining of the protein bands showed a high purity of the commercial HBc/sAg (FIG. 1). These antigens were used to test vaccines of the invention.

To prepare antigen-nanocomplexes, the HBV core and/or surface antigens (HBc/sAg) were mixed with γ-PGA to form a first charged polymer solution. Then, this solution was mixed with a second charged polymer solution (e.g., chitosan) in an appropriate ratio. The resulting HBc/sAg-nanocomplexes (HBc/s-NCs) were characterized with dynamic light scattering (DLS). Results of DLS show the sizes of HBc/s-NCs range from about 100 nm to about 800 nm, with average diameters in individual groups ranging from 344 nm to 573 nm (FIG. 2). The NC particles do not have a wide range of particle size variations, as evidenced by the low polydispersity index (PdI) determined by dynamic light scattering (DLS), and the zeta-potentials of these NCs were determined to range from about +30 mV to about +50 mV (FIG. 2).

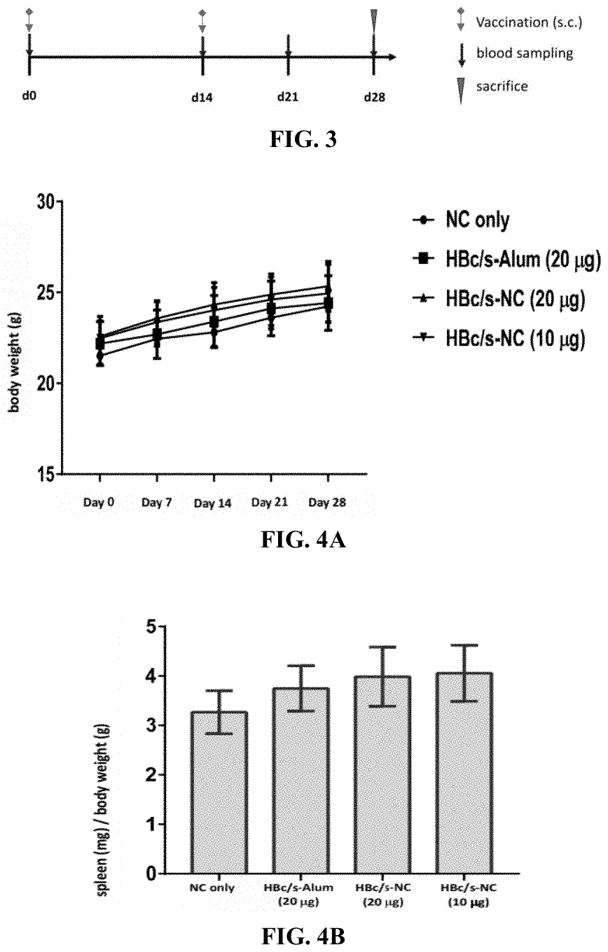

These antigen-NCs were tested for their abilities to elicit immune responses. The HBc/s-NCs were tested at two doses (10 μg and 20 μg) to assess the stimulation of antibody productions. The controls include NC only and HBc/s-Alum (20 μg/dose), which used a conventional adjuvant (alum). These vaccines were inoculated into C57BL/6 mice through subcutaneous (S.C.) route at day 0. The blood samples were obtained at days 0, 14, 21 and 28, and the mice were sacrificed at day 28 (FIG. 3). The body weights of the mice in the test groups were monitored and were found to be stable and comparable to the control groups, suggesting that these vaccinations were safe (FIG. 4A). In addition, the mice were sacrificed on day 28, and the spleens were harvested. The ratios of spleen weights divided by body weights at day 28 did not have significant differences, confirming the safety (e.g., no significant inflammation) of these vaccines (FIG. 4B).

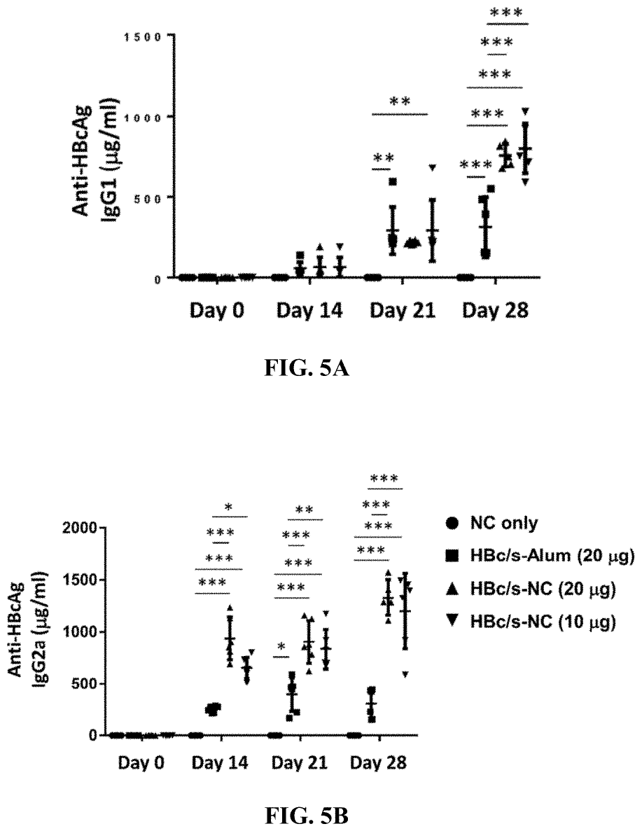

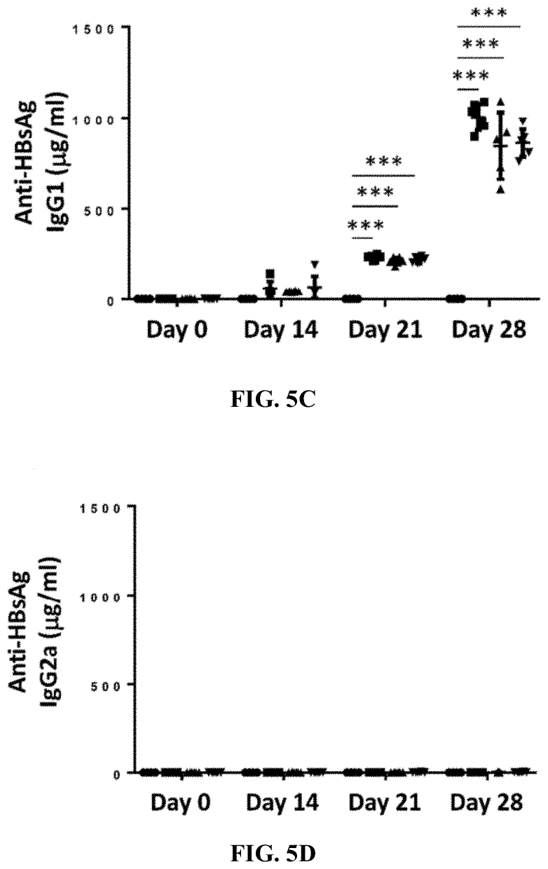

The abilities of these vaccines to induce immune responses were investigated by examining their impacts on various immune cells and the production of antibodies. In mice, Th1-dependent IFN-γ induces the production of IgG2a, while the Th2-dependent cytokine IL-4 stimulates the expression of IgG1. Therefore, IgG2a and IgG1 immunoglobulin isotypes can be used as markers for the polarization/activation of Th1 and Th2 lymphocytes, respectively. In the two dosage (10 μg/dose and 20 μg/dose) HBc/s-NC groups, serum HBcAg-specific IgG1 and IgG2a titers were both induced in the HBc/s-NC vaccinated mice (FIG. 5A and FIG. 5B).

As shown in FIG. 5A, the anti-HBV core antigen (anti-HBcAg) IgG1 titers appeared late and there were no significant differences between the nanocomplex vaccines and the alum vaccine for the first 21 days. By day 28, the nanocomplex vaccines of the invention (HBc/s-NC) at 10 μg or 20 μg induced significantly higher levels (about 2×) of anti-HBcAg IgG1 production, as compared with the conventional adjuvant (alum) one. These results indicate that the nanocomplex vaccines of the invention are significantly more effective in inducing the Th2 immune responses, as compared with the conventional vaccine (alum as adjuvant).

As shown in FIG. 5B, the nanocomplex vaccines of the invention (HBc/s-NC) at 10 μg or 20 μg induced significantly higher levels of anti-HBV core antigen (anti-HBcAg) IgG2a productions starting from day 14, as compare with the alum vaccine. By day 28, anti-HBcAg IgG2a productions induced by the nanocomplex vaccines of the invention (HBc/s-NC) were several folds (about 4 folds) higher than that induced by a vaccine with alum as an adjuvant. These results indicate that vaccines of the invention (nanocomplex vaccines) can activate higher IgGa2 antibody titers and elicit stronger cell-mediated immune responses (Th1 responses), as compared to a conventional vaccine using alum as an adjuvant. That nanocomplex vaccines of the invention are so much better in eliciting both Th2 responses and cell-mediated immune responses (Th1 responses) than a conventional vaccine is truly unexpected.

Nanocomplex vaccines (HBc/s-NC) of the invention can also induce anti-HBV surface antigen (anti-HBsAg) specific IgG1 titers. As shown in FIG. 5C, nanocomplex vaccines (HBc/s-NC) of the invention can induce strong anti-HBsAg IgG1 productions (Th2 immune responses) starting from day 21. The conventional vaccine with alum also produced strong anti-HBsAg IgG1 productions and there was no significant difference between vaccines of the invention and the alum vaccine (FIG. 5C). In contrast to the Th2 immune responses, no or little HBsAg-specific IgG2a titers (Th1 immune responses) were induced by vaccines of the invention or the alum vaccine (FIG. 5D).

The above results show that nanocomplexes of HBV core antigen (HBcAg) can polarize/activate both Th1 and Th2 cells, suggesting that the nanocomplex vaccines having HBV core antigen can enhance both humoral and cellular immune responses. On the other hand, the nanocomplexes of HBV surface antigen (HBsAg) only polarize/activate Th2 cells, suggesting that these vaccines can enhance humoral immune responses. More importantly, the nanocomplex vaccines of the invention are dramatically more effective in the polarization/activation of the T cells, as compared with a conventional adjuvant (e.g., alum) with the same antigens.

To further examine the HBc/s-NC induced immune responses and their potentials to change the immune tolerance status in chronic HBV patients, we investigated the effects of these vaccines using an animal model—i.e., by inoculating HBc/s-NC into AAV/HBV infected C57BL/6 mice, which were generated by Prof. Mi-Hua Tao (Institute of Biomedical Sciences, Academia Sinica, Taiwan). This HBV-carrier mouse model was developed by hydrodynamic injection (HDI) of the pAAV/HBV1.2 plasmid into C57BL/6 mice. (Huang et al., Proc. Natl. Acad. Sci. U.S.A. 2006 Nov. 21; 103(47):17862-17867).

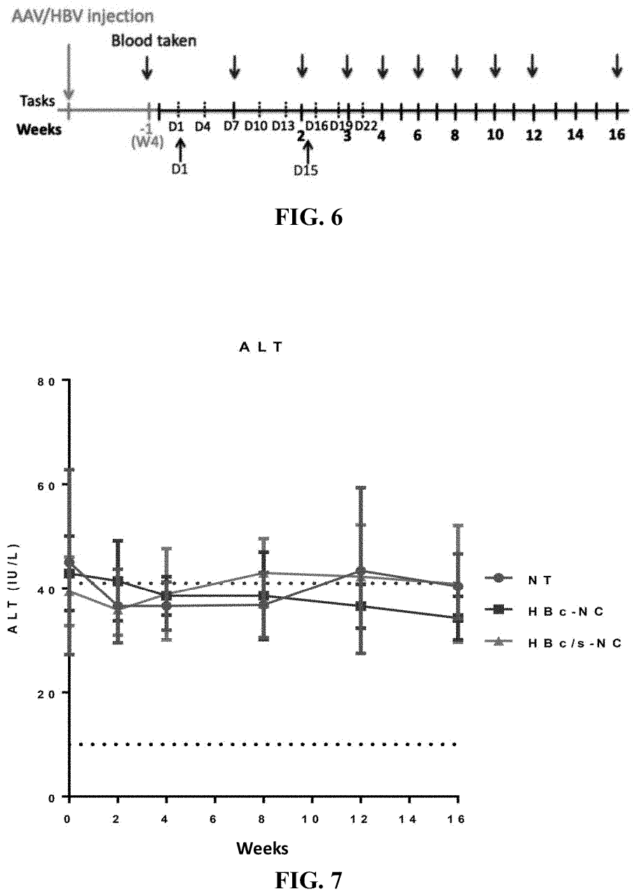

FIG. 6 shows an experimental protocol for the animal model study. At week −4, AAV/HBV were injected into mice through an intravenous (i.v.) route (tail vein) to generate HBV-carrier mouse model. Saline (non-treatment, NT), two doses of 10 μg/dose HBc-NC, or 10 μg/dose HBc/s-NCs were vaccinated through s.c. route at day 1 (week 0) and day 15. The blood samples were collected at day −1, 7, 14, every 2 weeks up to week 12, and week 16. The effects of these vaccines on HBV infections were assessed by several makers of liver health and HBV status.

First, mouse liver health is monitored by assessing alanine transaminase (ALT) and bilirubin levels in blood. ALT is a liver enzyme that is released in the blood when liver cells are damaged. Bilirubin comes from the breakdown of red blood cells and is excreted by the liver. High bilirubin levels can indicate a problem with the liver. FIG. 7 shows the ALT levels and FIG. 8 shows the bilirubin levels of the mice during the testing periods. These levels in the HBc/s-NC and HBc/s-NCs vaccinated mice are similar to those of the saline-treated control group (NT), suggesting that the HBc/s-NC and HBc/s-NCs vaccines are non-toxic to livers.

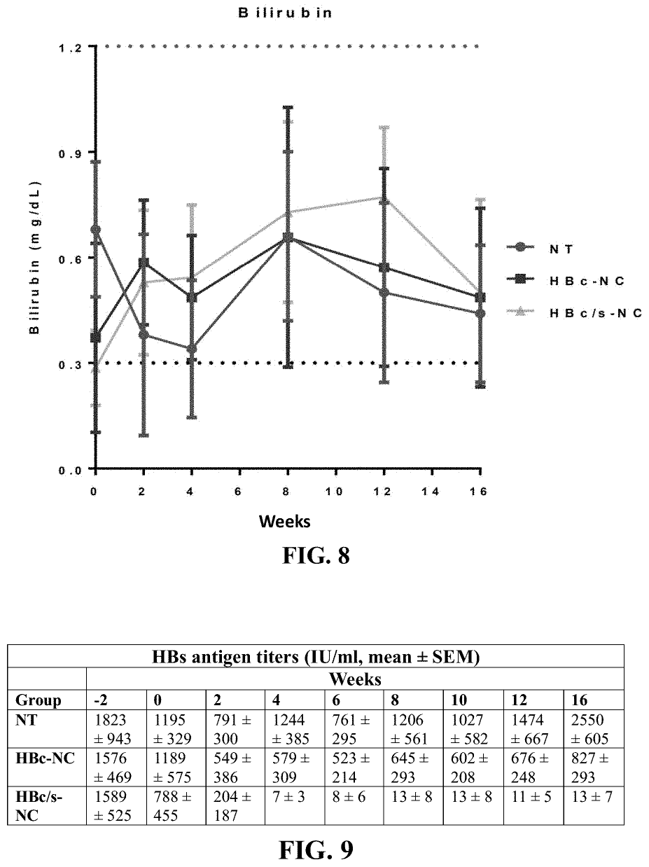

To investigate the therapeutic potentials of these nanocomplex vaccines, we evaluated the efficacy of HBc/s-NCs in the treatment of chronic HBV mice, by assessing HBs/eAg loss and HBsAb seroconversion. FIG. 9 summarizes the results and statistic data. The HBcAg-NC vaccinated group has slightly reduced HBsAg titers starting from week 2 and this reduced level is maintained over the course. The HBc/sAg-NC vaccinated group has significantly reduced HBsAg titers within 2 weeks and the HBsAg titers were almost eliminated starting from week 4 and maintained throughout the test duration. This result indicates that HBc/sAg-NC vaccines of the invention can eliminate HBsAg from an infected subject.

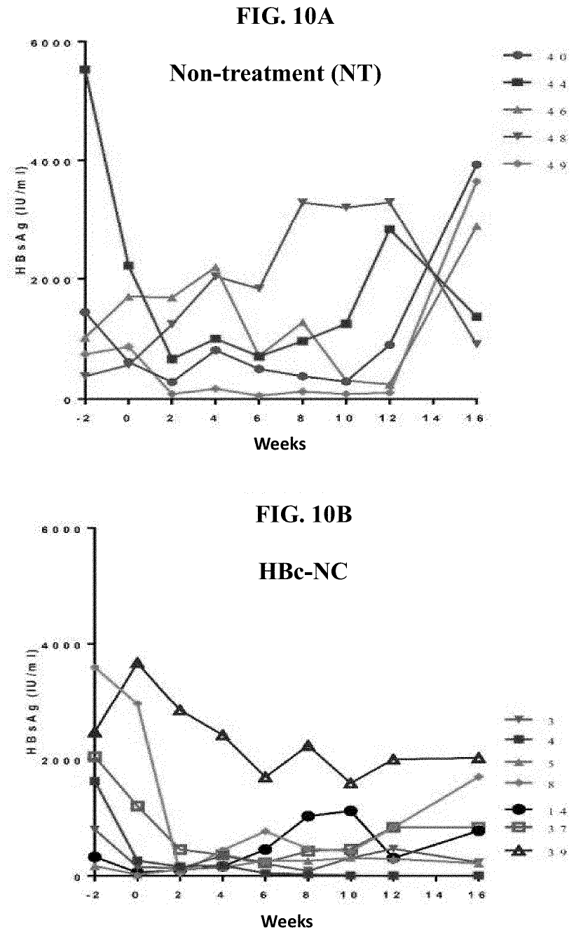

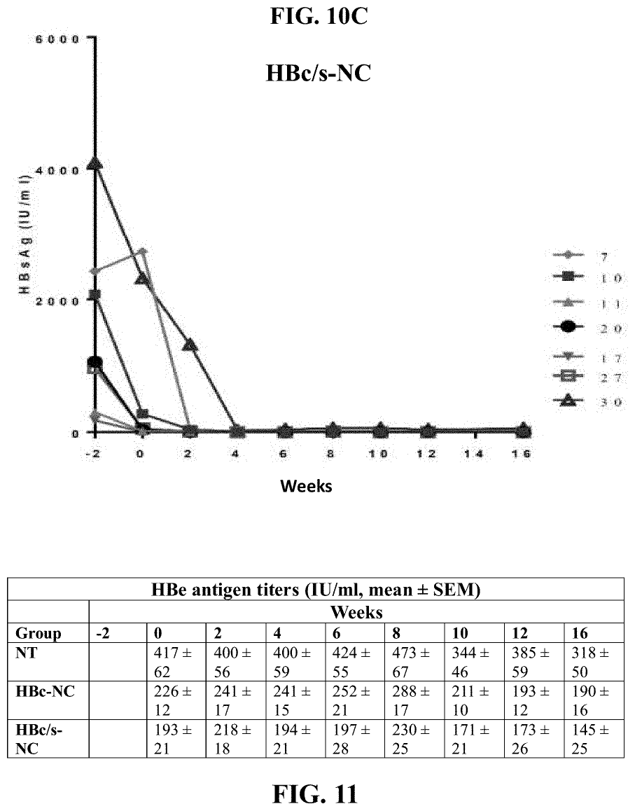

FIGS. 10A-10C show individual change curves of HBsAg for the control (non-treatment, NT), HBc-NC, and HBc/s-NC groups, respectively. As compared to the non-treatment group (FIG. 10A), HBc-NC (core antigen only) vaccinations resulted in substantial reduction in HBsAg titers, even though these mice still have lower levels of HBsAg over the test duration (FIG. 10B). In comparison, HBc/s-NC (core and surface antigens) vaccines caused sharp drops or complete elimination of HBsAg titers by week 4 and maintained at almost undetectable levels during the entire test duration (FIG. 10C). These results indicate that while HBc-NC vaccines are effective in substantially reducing the HBsAg titers, and the HBs/c-NC vaccines are much more effective in achieving the elimination of HBsAg.

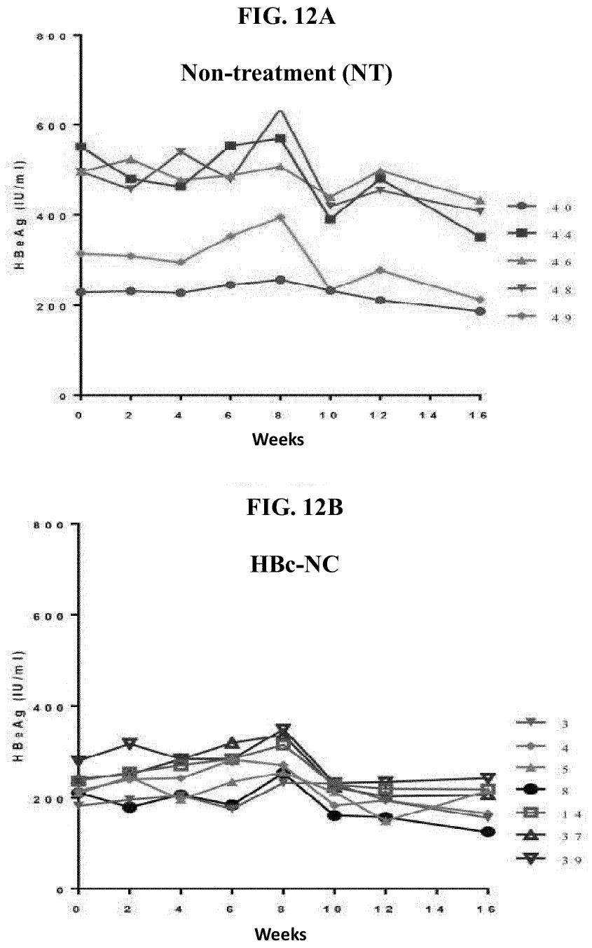

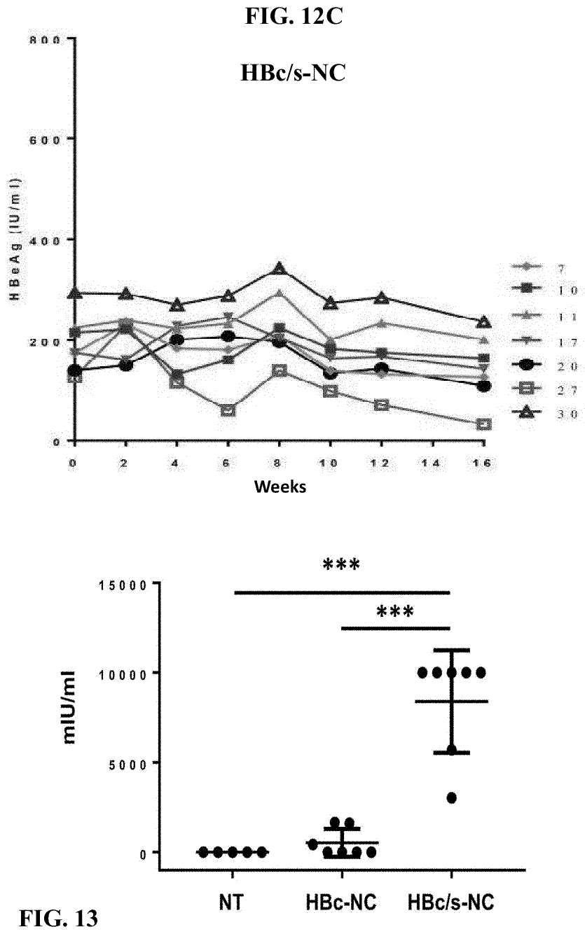

The presence of HBcAg and HBeAg proteins is an indication of viral replication. Thus, the presence of HBeAg in the serum of patients can serve as a marker of active replication in chronic hepatitis. We also investigated the effects of the NC vaccines of the invention on HBV replication. As shown in FIG. 11, the vaccinated mice showed slow changes in the HBeAg titers, decreasing at slow rates until week 16. FIGS. 12A-12C show individual change curves of HBeAg titers for the control (non-treatment, NT), HBc-NC, and HBc/s groups, respectively. The slow decreases in the vaccine treatment groups suggest that these vaccines gradually suppress HBV replications.

Antibody productions after inoculations with NC only, HBc/s-Alum, HBc-NC, 20 μg/dose HBc/s-NCs, or 10 μg/dose HBc/s-NCs were also assessed. A non-treatment (NT) group was used as a control. FIG. 13 shows exemplary HBsAg specific IgG serum levels at week 10 after vaccinations with HBc-NC and HBc/s-NC, as compared with no-treatment control (NT). These results show that the HBc/s-NC vaccines stimulated a high level of HBsAb, while the HBc-NC vaccines only induced a low-level production of the antibody.

Based on the above results, HBc/s-NCs of the invention can elicit high-level production of antibodies against HBsAg, a rapid elimination of HBsAg, and a slow decrease of HBeAg in mouse sera. These combined phenomena are indications of HBsAg seroconversion. The fact that HBc/s-NC vaccines of the invention can induce HBsAg seroconversion suggests that HBc/s-NC vaccines of the invention can overcome the immune tolerance caused by chronic HBV infection to re-establish the host immunity, resulting in a functional cure. Based on these serological markers, HBc/s-NCs vaccinations of the invention can rescue the chronic HBV infection at least into a functional cure, as evidenced by HBsAg loss and an undetectable level of HBV DNA in serum.

Embodiments of the invention will be further illustrated with specific examples and experimental details. One skilled in the art would appreciate that these examples are for illustration only and are not intended to limit the scope of the invention because other modifications and variations are possible without departing from the scope of the invention.

### EXAMPLES

### 1. Analysis of the Purity of HBc/s Proteins.

15% acrylamide gel is used to separate HBc/s antigens with other impurities. Proteins are divided into reduction and non-reduction groups. Reduction of Ags is accomplished with 2-ME or DTT with boiling. After electrophoresis, protein bands in gel are stained with Coomassie blue and distained with ddH₂O.

### 2. The Preparation and Characterization of HBc-NC or HBc/s-NC.

In accordance with embodiments of the invention, HBV core antigen nanocomplexes (HBc-NCs), HBV surface antigen nanocomplexes (HBs-NCs), or HBV core and surface antigens nanocomplexes (HBc/s-NCs) can be prepared in the following manner, or any similar manner: forming a first solution of the antigen and γ-Polyglutamic acid (γ-PGA), preparing a second solution containing chitosan, and then adding the second solution to the first solution. Any suitable γ-PGA may be used. For example, in preferred embodiments, the γ-PGA may have a weight-averaged molecular weight (M.W.) of about 200 kDa or less. Similarly, any suitable chitosan may be used. In preferred embodiments, chitosan may have a weight-averaged molecular weight (M.W.) of about 10-100 kDa. In addition, chitosan may have any degree of deacetylation, for example 0-100% deacetylation, preferably 50-100% deacetylation, more preferably 75-95% deacetylation. Any suitable concentrations of the antigens, γ-PGA, and chitosan may be used. For example, the concentrations for the antigens may be 2 to 0.5 mg/ml, the concentrations for chitosan may be 20 to 30 mg/ml, and the concentrations for γ-PGA may be 5 to 20 mg/ml.

As an example, a first solution is prepared with γ-Polyglutamic acid (γ-PGA; w/v=1% in ddH₂O; M.W. range=about 200 kDa or less) and a predetermined amount of HBcAg and/or HBsAg. A second solution is prepared with chitosan in 1% acetic acid (w/v=2.5% chitosan, M.W. range=about 10-100 kDa). Add the second solution (chitosan solution) to the first solution (γ-PGA with HBcAg and/or HBsAg) to form nanocomplexes (NCs). NCs were stored at 4° C. overnight for the stability tests. The sizes, zeta-potentials, and polydispersity index (PdI) were determined with Malvern Zetasizer Nano Series (Zetasizer Nano ZS, Malvern Panalytical Ltd., U.K.).

### 3. Mice.

All animal studies were conducted under specific pathogen-free conditions. In antibody induction experiments, 22 six- to eight-week-old male C57BL/6 mice, purchased from National Laboratory Animal Center, Taiwan, were divided into 4 groups: NC only (4 mice), 20 μg/dose HBc/s-Alum (6 mice), 20 μg/dose HBc/s-NC (6 mice), and 10 μg/dose HBc/s-NC (6 mice). Mice were inoculated with these vaccines at days 0 and 14. The mice were blood sampled at days 0, 14, 21 and 28, and weighted weekly. After mice were sacrificed at day 28, spleens were removed and weighed to evaluate the systemic inflammation. HBc/sAbs levels in sera were determined with ELISA.

In AAV/HBV mice model, six- to eight-week-old male C57BL/6 mice were purchased from National Laboratory Animal Breeding and Research Center, Taiwan. To establish persistent HBV gene expression in the liver of immunocompetent mice, we used the hepatotropic AAV serotype 8 vector (AAV8), which has a high liver transduction rate, to deliver the HBVp-genome. This recombinant virus carries 1.3 copies of the HBV genome (genotype D) with a point mutation on polymerase and is packaged in AAV serotype 8 (AAV8) capsids. AAV/HBVp-vector produces all HBV proteins but does not produce infectious HBV particles. Mice are intravenously injected with 2×10¹⁰ AAV/HBVp-suspended in 100 μl saline through tail vein. Subsequently, serum HBsAg and HBeAg levels are measured to confirm the state of HBV persistence.

### 4. Enzyme-Linked Immunosorbent Assay (ELISA) for HBV Antigens and Specific Antibodies Detection.

Standard IgG1/2a or HBc/s proteins are coated on 96 well plate at appropriate concentrations and kept at 4° C. overnight. Mouse sera are serially diluted and added into each well to incubate for 2 h, at RT. Anti-IgG1 or anti-IgG2a antibodies conjugated HRP are added into wells and incubate for 1 h, at RT. Add TMB subtract to produce color products, and stop the reaction by addition of 2N HCl. Levels of HBsAg, HBeAg, and anti-HBs antibodies in mouse sera were measured using an Elecsys Systems electrochemiluminescence kit and a Cobas analyzer (E601 module, Roche Diagnostics GmbH).

### 5. Serum Levels of ALT and T-BIL

Alanine aminotransferase (ALT) activity and total bilirubin (T-BIL) levels in sera were measured using Vitros Chemistry Products ALT slides or T-BIL slides, respectively, using a Vitros 950 chemical analyzer (Johnson & Johnson, Rochester, N.Y.).

Embodiments of the invention have been described with referenced to specific examples. One skilled in the art would appreciate that these examples are for illustration only and that other modifications and variations are possible without departing from the scope of the invention. Accordingly, the scope of protection of the invention should be determined by the attached claims.

1. A hepatitis B virus (HBV) vaccine, comprising: an HBV core antigen (HBcAg) and/or HBV surface antigen (HBsAg) formulated in nanocomplexes.

2. The HBV vaccine according to claim 1, wherein the vaccine comprises both HBV core antigen (HBcAg) and HBV surface antigen (HBsAg).

3. The HBV vaccine according to claim 1, wherein the nanocomplexes comprise γ-polyglutamic acid (γ-PGA) and chitosan.

4. The HBV vaccine according to claim 1, wherein the nanocomplexes are prepared by a first solution containing the HBV core antigen (HBcAg) and/or HBV surface antigen (HBsAg) and the γ-polyglutamic acid (γ-PGA), forming a second solution containing chitosan, and adding the second solution into the first solution or adding the first solution into the second solution.

5. The HBV vaccine according to claim 4, wherein a concentration of HBcAg and/or HBc/sAg in the first solution is 2-0.5 mg/ml and a concentration of γ-PGA in the first solution is 5-20 mg/ml, and wherein a concentration of chitosan in the second solution is 20-30 mg/ml.

6. The HBV vaccine according to claim 1, wherein the nanocomplexes have zeta potentials of about +30 mV to about +50 mV.

7. A method for treating HBV infection, comprising administering to a subject in need thereof an effective amount of the HBV vaccine according to claim 1.

8. The method according to claim 7, wherein the subject has chronic HBV infection.

9. The HBV vaccine according to claim 2, wherein the nanocomplexes comprise γ-polyglutamic acid (γ-PGA) and chitosan.

10. The HBV vaccine according to claim 2, wherein the nanocomplexes are prepared by a first solution containing the HBV core antigen (HBcAg) and/or HBV surface antigen (HBsAg) and the γ-polyglutamic acid (γ-PGA), forming a second solution containing chitosan, and adding the second solution into the first solution or adding the first solution into the second solution.

11. The HBV vaccine according to claim 3, wherein the nanocomplexes are prepared by a first solution containing the HBV core antigen (HBcAg) and/or HBV surface antigen (HBsAg) and the γ-polyglutamic acid (γ-PGA), forming a second solution containing chitosan, and adding the second solution into the first solution or adding the first solution into the second solution.

12. The HBV vaccine according to claim 2, wherein the nanocomplexes have zeta potentials of about +30 mV to about +50 mV.

13. The HBV vaccine according to claim 3, wherein the nanocomplexes have zeta potentials of about +30 mV to about +50 mV.

14. The HBV vaccine according to claim 4, wherein the nanocomplexes have zeta potentials of about +30 mV to about +50 mV.

15. The HBV vaccine according to claim 5, wherein the nanocomplexes have zeta potentials of about +30 mV to about +50 mV.

16. The method for treating HBV infection according to claim 7, wherein the vaccine comprises both HBV core antigen (HBcAg) and HBV surface antigen (HBsAg).

17. The method for treating HBV infection according to claim 7, wherein the nanocomplexes comprise γ-polyglutamic acid (γ-PGA) and chitosan.

18. The method for treating HBV infection according to claim 7, wherein the nanocomplexes are prepared by a first solution containing the HBV core antigen (HBcAg) and/or HBV surface antigen (HBsAg) and the γ-polyglutamic acid (γ-PGA), forming a second solution containing chitosan, and adding the second solution into the first solution or adding the first solution into the second solution.

19. The method for treating HBV infection according to claim 7, wherein a concentration of HBcAg and/or HBc/sAg in the first solution is 2-0.5 mg/ml and a concentration of γ-PGA in the first solution is 5-20 mg/ml, and wherein a concentration of chitosan in the second solution is 20-30 mg/ml.

20. The method for treating HBV infection according to claim 7, wherein the nanocomplexes have zeta potentials of about +30 mV to about +50 mV.
